(19) United States
(12) Patent Application Publication
(10) Pub. No.: US 2010/0312057 A1
(43) Pub. Date: Dec. 9, 2010

(54) Illumination optical system for endoscopes

(76) Inventor: Mitsujiro Konno, Tokyo (JP)

Correspondence Address: KENYON & KENYON LLP, ONE BROADWAY, NEW YORK, NY, 10004, US

(21) Appl. No.: 12/802,516

(22) Filed: Jun. 7, 2010

Related U.S. Application Data
(63) Continuation of application No. PCT/JP2009/068882, filed on Nov. 5, 2009.

(30) Foreign Application Priority Data
Nov. 11, 2008 (JP) 2008-288915

(51) Int. Cl. A61B 1/06

(52) U.S. Cl. 600/162; 600/178

(57) ABSTRACT

An illumination optical system for endoscopes, which is used by combining the llumination optical system with an objective optical system, where the objective optical system can observe a predetermined area at least ranging from the lateral direction to the backward direction with respect to the longitudinal direction of an endoscope and across the range of 180 degrees or more in the circumferential direction of the endoscope, and which is provided with an illumination optical system capable of radiating light to the predetermined area which can be observed by the objective optical system, is formed in such a way that illumination light becomes central darkening along the longitudinal direction of the endoscope and across the range of 180 degrees in the circumferential direction of the endoscope which can be observed by the objective optical system and that the illumination optical system has a luminous intensity distribution characteristic in which the central-darkening illumination light is radiated to the vicinity of the lateral direction with respect to the longitudinal direction of the endoscope.

### BACKGROUND OF THE INVENTION

This invention relates to an illumination optical system for endoscopes, which is used for example, for an endoscope by which observations in the lateral and backward directions can be performed.

Conventional endoscopes, for example, which are used for detecting a lesion behind folds in a luminal object such as large intestine, include an endoscope by which it is possible to observe a predetermined area ranging from the lateral direction to the backward direction with respect to the longitudinal direction of the endoscope.

For example, endoscopes by which it is possible to observe a predetermined area ranging from the lateral direction to the backward direction with respect to the longitudinal direction of each of the endoscopes are disclosed by Japanese Patent Kokai No. 2002-65589 (FIG. 5), Japanese Patent Kokai No. 2004-33487 (FIG. 8), Japanese Patent Kokai No. 2005-319315 (FIG. 2), Japanese Patent Kokai No. Hei 7-191269 (FIG. 1), Japanese Patent Kokai No. 2004-329700 (FIG. 1), and Japanese Patent Kokai No. 2003-164418 (FIG. 4), respectively.

FIG. 1 is a conceptual view showing an area observed by an observation system (objective optical system) and an area of illumination light radiated by an illumination system in the conventional case of observing a luminal object using an endoscope by which it is possible to observe a predetermined area ranging from the lateral direction to the backward direction with respect to the longitudinal direction of the endoscope.

In the case where illumination light is radiated from an area light source to a luminal object, if the luminal object is regarded as a plane-shaped object, then the illuminance of light radiated to the plane is affected by the cosine fourth law.

### SUMMARY OF THE INVENTION

An illumination optical system for endoscopes according to the present invention which is used by combining the illumination optical system with an objective optical system, where the objective optical system makes it possible to observe a predetermined area at least ranging from the lateral direction to the backward direction with respect to the longitudinal direction of an endoscope and across the range of 180 degrees or more in the circumferential direction of the endoscope, and which is provided with an illumination optical system capable of radiating light to the predetermined area which can be observed by the objective optical system, is formed in such a way that: illumination light becomes central darkening along the longitudinal direction of the endoscope and across the range of 180 degrees in the circumferential direction of the endoscope which can be observed by the objective optical system; and the illumination optical system has a luminous intensity distribution characteristic in which the central-darkening illumination light is radiated to the vicinity of the lateral direction with respect to the longitudinal direction of the endoscope.

Also, in an illumination optical system for endoscopes according to the present invention, it is preferred that the luminous intensity distribution characteristic along the longitudinal direction of the endoscope satisfies the following condition (1) in the illumination optical system:

I₀≦Iθ (1)

where: I denotes the intensity of light which is radiated by the illumination optical system with a luminous intensity distribution for a spherical object at a predetermined angle to the direction perpendicular to the longitudinal direction of the endoscope within the range of the field of view which can be observed by the objective optical system, on the basis of a light radiation plane of the illumination optical system; I₀ denotes the intensity of light which is radiated at an angle of zero degrees to the direction perpendicular to the longitudinal direction of the endoscope by the illumination optical system with a luminous intensity distribution for a spherical object; and Iθ denotes the intensity of light which is radiated backward at an angle of θ degrees to the direction perpendicular to the longitudinal direction of the endoscope by the illumination optical system with a luminous intensity distribution for a spherical object.

Also, it is preferred that: an illumination optical system for endoscopes according to the present invention further comprises a second illumination optical system which is used by combing the second illumination optical system with an objective optical system by which forward observation can be performed, and which can radiate light forward; and the second illumination optical system has a luminous intensity distribution characteristic in which illumination light does not become central darkening along the longitudinal direction of the endoscope.

Also, in an illumination optical system for endoscopes according to the present invention, it is preferred that a luminous intensity distribution characteristic in the longitudinal direction of the endoscope is different from a luminous intensity distribution characteristic in the circumferential direction of the endoscope.

Also, in an illumination optical system for endoscopes according to the present invention, it is preferred that the luminous intensity distribution characteristic along the longitudinal direction of the endoscope satisfies the following condition (1′) in the illumination optical system:

I₀≦I₄₀ (1′)

where: I denotes the intensity of light which is radiated by the illumination optical system with a luminous intensity distribution for a spherical object at a predetermined angle to the direction perpendicular to the longitudinal direction of the endoscope within the range of the field of view which can be observed by the objective optical system, on the basis of a light radiation plane of the illumination optical system; I₀ denotes the intensity of light which is radiated at an angle of zero degrees to the direction perpendicular to the longitudinal direction of the endoscope by the illumination optical system with a luminous intensity distribution for a spherical object; and I₄₀ denotes the intensity of light which is radiated backward at an angle of 40 degrees to the direction perpendicular to the longitudinal direction of the endoscope by the illumination optical system with a luminous intensity distribution for a spherical object.

Also, in an illumination optical system for endoscopes according to the present invention, it is preferred that the luminous intensity distribution characteristic along the longitudinal direction of the endoscope satisfies the following condition (1″) in the illumination optical system:

I₀≦I₅₀ (1″)

where: I denotes the intensity of light which is radiated by the illumination optical system with a luminous intensity distribution for a spherical object at a predetermined angle to the direction perpendicular to the longitudinal direction of the endoscope within the range of the field of view which can be observed by the objective optical system, on the basis of a light radiation plane of the illumination optical system; I₀ denotes the intensity of light which is radiated at an angle of zero degrees to the direction perpendicular to the longitudinal direction of the endoscope by the illumination optical system with a luminous intensity distribution for a spherical object; and I₅₀ denotes the intensity of light which is radiated backward at an angle of 50 degrees to the direction perpendicular to the longitudinal direction of the endoscope by the illumination optical system with a luminous intensity distribution for a spherical object.

Also, in an illumination optical system for endoscopes according to the present invention, it is preferred that the illumination optical system comprises a light source unit having a luminous intensity distribution characteristic in which illumination light becomes central darkening, a light guide which light from the light source unit enters, and an reflection means which is approximately shaped like a ring and is provided with a reflection surface along the circumferential direction of the endoscope, where the reflection surface reflects light emerging from the light guide, at the angle of reflection of 45 degrees or more.

Also, in an illumination optical system for endoscopes according to the present invention, it is preferred that the illumination optical system comprises a light source unit having a luminous intensity distribution characteristic in which illumination light becomes central darkening, a light guide which light from the light source unit enters, and an reflection means which is approximately shaped like a ring and is provided with a reflection surface along the circumferential direction of the endoscope, where the reflection surface reflects light emerging from the light guide, at the angle of reflection of approximately 50 degrees.

Also, it is preferred that an illumination optical system for endoscopes according to the present invention is provided with a means for making the angle-of-incidence characteristics of light rays from the light source unit incident on the light guide the same characteristic.

Also, in an illumination optical system for endoscopes according to the present invention, it is preferred that the means for making the angle-of-incidence characteristics of the light rays the same characteristic is a concave lens which is arranged on the entrance side of the light guide.

Also, in an illumination optical system for endoscopes according to the present invention, it is preferred that the light source unit is provided with a field-of-view mask blocking a light ray the angle of incidence of which is small.

Also, in an illumination optical system for endoscopes according to the present invention, it is preferred that the illumination optical system comprises a variable magnification optical system having a function of removing pupil aberration.

Also, it is preferred that an illumination optical system for endoscopes according to the present invention is provided with a light diffusing means on the reflection side of the reflection surface.

Also, in an illumination optical system for endoscopes according to the present invention, it is preferred that the reflection surface has the diffusion effect while the luminous intensity distribution characteristic in which illumination light becomes central darkening along the longitudinal direction of the endoscope is held.

Also, in an illumination optical system for endoscopes according to the present invention, it is preferred that the reflection surface is formed as a convex surface.

Also, in an illumination optical system for endoscopes according to the present invention, it is preferred that the illumination optical system is provided with a lens along the circumferential direction of the endoscope, where the lens is provided with a light blocking means in the central portion of the end surface on the exit side thereof.

Also, in an illumination optical system for endoscopes according to the present invention, it is preferred that the illumination optical system is provided with a light guide and a condenser lens along the circumferential direction of the endoscope, where the light guide is provided with a light blocking means in the central portion of the end plane on the exit side thereof and the condenser lens is arranged on the exit side of the light guide.

Also, in an illumination optical system for endoscopes according to the present invention, it is preferred that the illumination optical system is provided with a lens along the circumferential direction of the endoscope, where the lens has a plane having two optical powers and the luminous intensity distribution characteristics of the plane having the two optical powers are superposed so that the lens has a luminous intensity distribution characteristic in which illumination light becomes central darkening along the longitudinal direction of the endoscope.

Also, in an illumination optical system for endoscopes according to the present invention, it is preferred that the illumination optical system is provided with a reflection surface along the circumferential direction of the endoscope, where the reflection surface is provided with a film and the reflection characteristic of the film varies depending on angle of incidence.

Also, in an illumination optical system for endoscopes according to the present invention, it is preferred that the illumination optical system is provided with a plurality of reflection surfaces along the circumferential direction of the endoscope and reflection surfaces adjacent to each other in a plurality of the reflection surfaces differ from each other in angle of reflection so that the luminous intensity distribution characteristic along the longitudinal direction of the endoscope by the reflection surfaces adjacent to each other becomes a luminous intensity distribution characteristic in which illumination light becomes central darkening.

Also, in an illumination optical system for endoscopes according to the present invention, it is preferred that the illumination optical system is provided with a plurality of LED light sources along the circumferential direction of the endoscope and each of the LED light sources is provided with a light attenuating means in the middle area in front of the light emitting means along the circumferential direction of the endoscope, where the light attenuating means reduces a light ray having a small angle in the luminous intensity distribution.

Also, in an illumination optical system for endoscopes according to the present invention, it is preferred that: the illumination optical system comprises two illumination systems which are arranged along the longitudinal direction of the endoscope and illuminate the lateral surrounding area of the endoscope; and the light intensities of the back-side illumination system of the two illumination systems illuminating the lateral surrounding area of the endoscope are intensified in order to make luminous intensity distribution in which illumination light becomes central darkening.

Also, in an illumination optical system for endoscopes according to the present invention, it is preferred that: the illumination optical system comprises a light source unit and a light guide which light from the light source unit enters; and the light guide has one end plane on the entrance side and the exit-side end plane of the light guide branches into a plurality of end planes, and one of a plurality of the end planes is used for forward radiation of illumination light and the other planes of a plurality of the end planes are used for radiation of illumination light in the direction perpendicular to the longitudinal direction of the endoscope or backward radiation of illumination light.

Also, in an illumination optical system for endoscopes according to the present invention, it is preferred that the illumination optical system is provided with a plurality of light sources along the circumferential direction of the endoscope, where the light sources have a luminous intensity distribution characteristic in which illumination light becomes central darkening, in such a way that the luminous intensity distribution to characteristic in the circumferential direction of the endoscope retains a predetermined luminous intensity by the superposition of the luminous intensities of light sources adjacent to each other in a plurality of the light sources.

Also, in an illumination optical system for endoscopes according to the present invention, it is preferred that the optical power in the longitudinal direction of the endoscope differs from the optical power in the circumferential direction of the endoscope in the illumination optical system.

Also, in an illumination optical system for endoscopes according to the present invention, it is preferred that the illumination optical system comprises a light guide having an end plane the diameter corresponding to the longitudinal direction of the endoscope of which differs from the diameter of the end plane corresponding to the circumferential direction of the endoscope in the illumination optical system.

Also, in an illumination optical system for endoscopes according to the present invention, it is preferred that the illumination optical system comprises a diffuser element which controls a luminous intensity distribution characteristic.

Also, in an illumination optical system for endoscopes according to the present invention, it is preferred that the illumination optical system is provided with a plurality of sets of light sources along the longitudinal direction of the endoscope, where each of the sets of the light sources consists of the light sources which have a Lambertian characteristic and are arranged along the circumferential direction of the endoscope, in such a way that the light sources in sets of the light sources which are adjacent to each other are staggered with the light sources of one of the sets adjacent to each other alternating with the light sources of the other of the sets adjacent to each other.

The features and advantages of the present invention will become apparent from the following detailed description of the embodiments when taken in conjunction with the accompanying drawings.

### BRIEF DESCRIPTION OF THE DRAWINGS

FIG. 1 is a conceptual view showing an area observed by an observation system (objective optical system) an area of radiation of illumination light by an illumination system in the case where a luminal object is observed by an endoscope which can observe a predetermined area ranging the lateral direction to the backward direction with respective to the longitudinal direction.

FIG. 2 is an explanatory view showing one example of an observation system (objective optical system) which is preferable for using an illumination optical system for endoscopes according to the present invention, in an endoscope capable of observing the backward area.

FIG. 3 is a graph showing the luminous intensity distribution characteristic of an illumination optical system which is commonly used for an endoscope.

FIG. 4 is an explanatory view showing the schematic formations of an illumination optical system for endoscopes according to a first embodiment of the present invention, (a) is a view showing one example of a light source unit which is used as one component for the illumination optical system for endoscopes according to the first embodiment, (b) is an explanatory view showing a state in which light beams from the light source unit shown in (a) are incident on the entrance-side end plane of a light guide which is used as one component for the illumination optical system for endoscopes according to the first embodiment, (c) is an important part-explaining view showing one example of variations of the illumination optical system for endoscopes according to the first embodiment, (d) is an important part-explaining view showing another example of variations of the illumination optical system for endoscopes according to the first embodiment, (e) is a sectional explanatory view showing one example of the reflection plane of a reflection means which is used as one component for the illumination optical system for endoscopes according to the first embodiment, and (f) is an explanatory view showing one example of variations of the reflection means shown in (e).

FIG. 5 is a graph showing one example of the luminous intensity distribution characteristic of the illumination optical system for endoscopes according to the first embodiment.

FIG. 6 is an explanatory view showing one example of the reflection means which is used as one component for the illumination optical system for endoscopes according to the first embodiment, (a) is a perspective view showing the whole formation of the reflection means, and (b) is the side view of (a) showing the positional relation between the reflection planes and the light guides.

FIG. 7 is an explanatory view showing the schematic formation of an important part of an illumination optical system for endoscopes according to a second embodiment of the present invention, (a) is a view showing one example of an illumination lens which is used as one component for the illumination optical system for endoscopes according to the second embodiment, (b) is a view showing one example of variations of the illumination optical system according for endoscopes according to the second embodiment, and (c) is a view showing another example of variations of the illumination optical system according for endoscopes according to the second embodiment.

FIG. 8 is an explanatory view showing the schematic formation of an important part for still another example of variations of the illumination optical system for endoscopes according to the second embodiment of the present invention.

FIG. 9 is an explanatory view showing the schematic formation of an important part for still another example of variations of the illumination optical system for endoscopes according to the second embodiment of the present invention.

FIG. 10 is an explanatory view showing the schematic formation of an important part for still another example of variations of the illumination optical system for endoscopes according to the second embodiment of the present invention.

FIG. 11 is a conceptual view showing one example of the positional relation between light sources and the angles of distribution of illumination light when an endoscope which is provided with the illumination optical system for endoscopes according to the present invention is viewed from the front.

FIG. 12 is an explanatory view showing the formation of an important part for an illumination optical system for endoscopes according to a third embodiment of the present invention.

FIG. 13 is an explanatory view showing one example of an illumination optical system for endoscopes according to a fifth embodiment of the present invention.

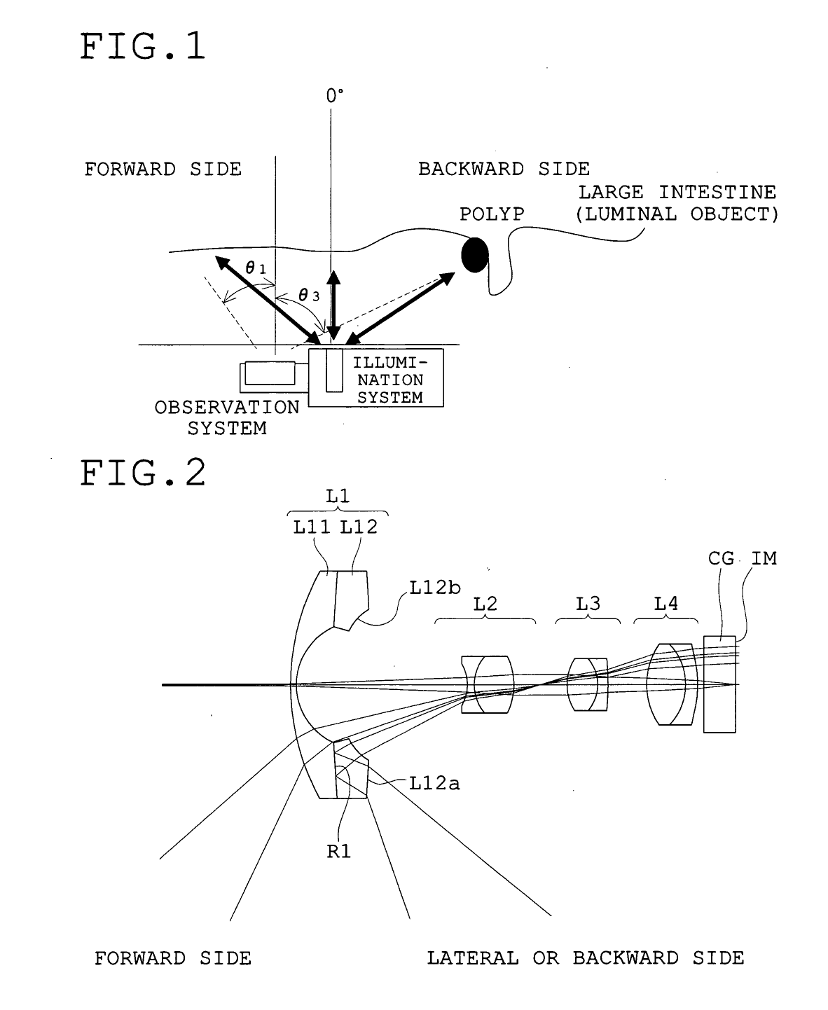

### DETAILED DESCRIPTION OF THE PREFERRED EMBODIMENTS

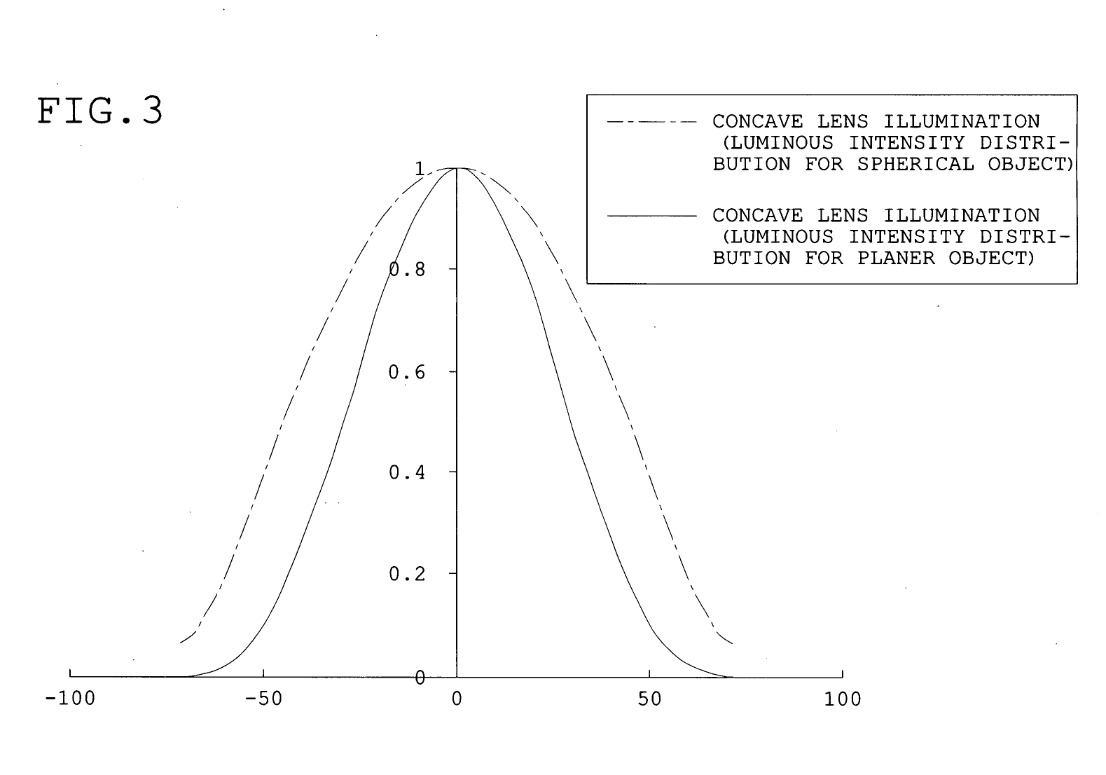

FIG. 2 is an explanatory view showing one example of an observation system (objective optical system) which is preferable for using an illumination optical system for endoscopes according to the present invention, in an endoscope capable of performing a backward observation. FIG. 3 is a graph showing the luminous intensity distribution characteristic of an illumination optical system which is commonly used for an endoscope. When the direction along the optical axis of the observation optical system in an endoscope is regarded as zero degrees, the horizontal axis in FIG. 3 indicates a viewing angle which is expressed as a negative value when the viewing angle to the optical axis is made forward on the basis of the direction along the optical axis and as a positive value when the viewing angle to the optical axis is made backward on the basis of the direction along the optical axis. The vertical axis in FIG. 3 indicates the proportion of light intensities when the light intensity in the direction along the optical axis is regarded as one.

In an observation system shown in FIG. 2, an objective lens L1 comprises a concave lens L11 the concave surface of which faces toward the image side, and a ring-shaped lens L12. The ring-shaped lens L12 includes an entrance surface L12a and an exit surface L12b on the image side. A ring-shaped reflection surface R1 is provided between the concave lens L11 and the ring-shaped lens L12. And, the observation system is formed in such a way that light rays which have come from the forward side and passed through the area of the effective diameter in the concave lens L11 and light rays which have come from a predetermined area ranging from the lateral direction to the backward direction, entered the image-side surface L12a of the ring-shaped lens L12 to have been reflected by the ring-shaped reflection surface R1, and emerged from the exit surface L12b form an image on the imaging plane IM of an imaging element, respectively. Besides, in FIG. 2, L2 to L4 denote a lens, and CG denotes a cover glass.

Besides, FIG. 2 shows merely an example of an observation system for an endoscope, which is preferable for using an illumination optical system for endoscopes according to the present invention and can perform a backward observation. Accordingly, any constitution of the observation system may be adopted if the observation system with the constitution can observe a predetermined area ranging from the lateral direction to the backward direction.

In an illumination optical system having the luminous intensity distribution characteristic as shown in FIG. 3, the intensity of light exiting in the direction along the optical axis of the illumination optical system is the highest on the basis of a position from which illumination light emerges, and, as a value on the horizontal axis in FIG. 3 is more distant from the center of the distribution characteristic shown in FIG. 3 which corresponds to the optical axis, the light intensity becomes lower according to the cosine fourth law.

In an illumination optical system for endoscopes according to the present invention, the constitutions as described in the following embodiments make it possible to decrease the luminous intensity of light which is radiated at a lateral viewing angle of the observation system in the vicinity of zero degrees as well as to increase the luminous intensity of light which is radiated at a backward viewing angle of the observation system in the vicinity of 40 degrees.

### The First Embodiment

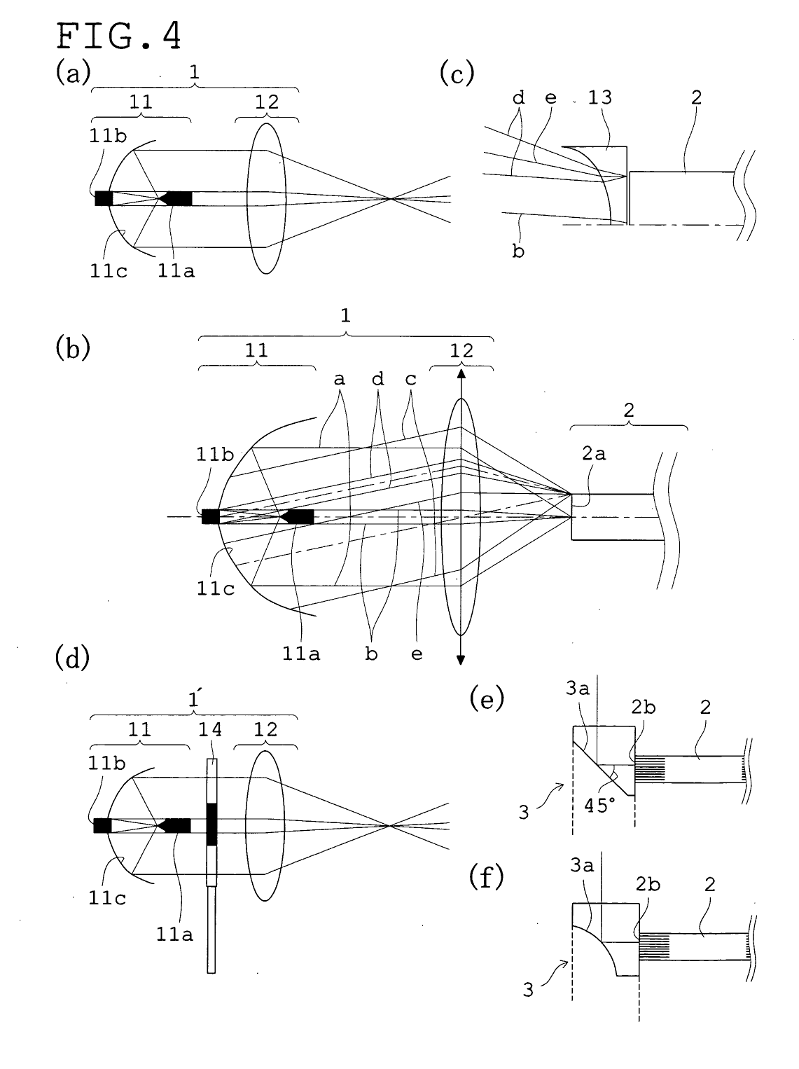

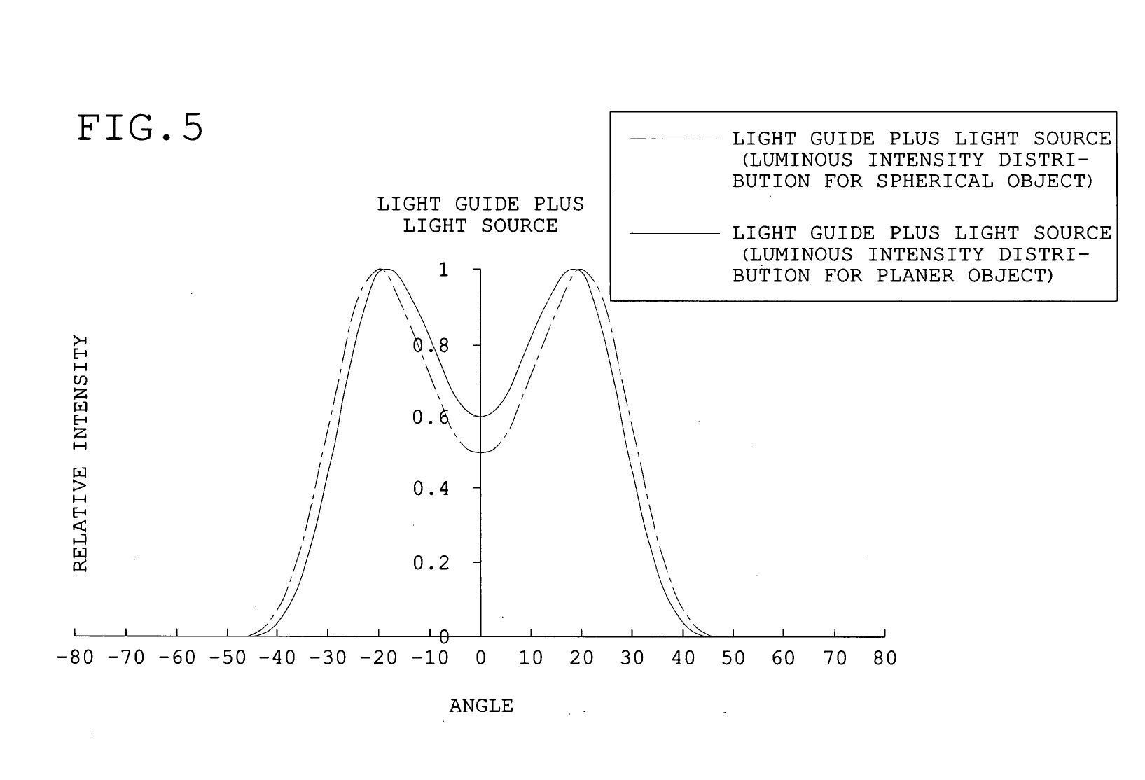

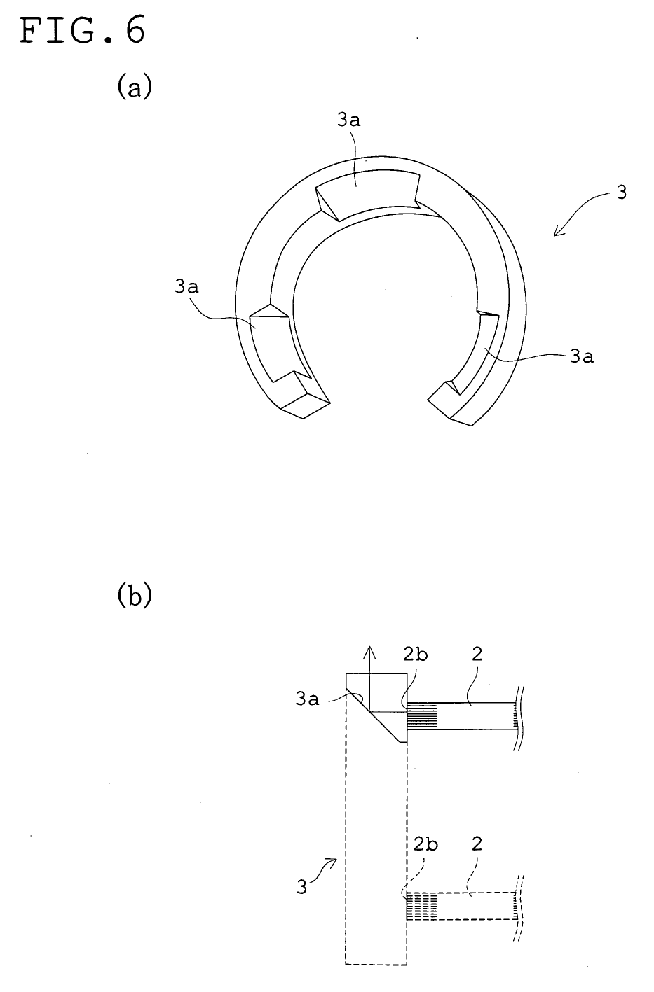

FIG. 4 is an explanatory view showing the schematic formations of an illumination optical system for endoscopes according to a first embodiment of the present invention, (a) is a view showing one example of a light source unit which is used as one component for the illumination optical system for endoscopes according to the first embodiment, (b) is an explanatory view showing a state in which light beams from the light source unit shown in (a) are incident on the entrance-side end plane of a light guide which is used as one component for the illumination optical system for endoscopes according to the first embodiment, (c) is an important part-explaining view showing one example of variations of the illumination optical system for endoscopes according to the first embodiment, (d) is an important part-explaining view showing another example of variations of the illumination optical system for endoscopes according to the first embodiment, (e) is a sectional explanatory view showing one example of the reflection plane of a reflection means which is used as one component for the illumination optical system for endoscopes according to the first embodiment, and (f) is an explanatory view showing one example of variations of the reflection means shown in (e). FIG. 5 is a graph showing one example of the luminous intensity distribution characteristic of the illumination optical system for endoscopes according to the first embodiment. FIG. 6 is an explanatory view showing one example of the reflection means which is used as one component for the illumination optical system for endoscopes according to the first embodiment, (a) is a perspective view showing the whole formation of the reflection means, and (b) is the side view of (a) showing the positional relation between the reflection planes and the light guides.

The illumination optical system according to the first embodiment comprises a light source unit 1 (refer to FIGS. 4(a) and (b)), a light guide 2 (refer to FIG. 4(b)) which light from the light source unit 1 enters, and a reflection means 3 (refer to FIG. 4(e) and FIG. 6) which reflects light from the light guide 2. The light source unit 1 includes a light source 11 and a condenser lens 12 which concentrates light from the light source.

In this case, the illumination optical system for endoscopes according to the first embodiment uses as the light source 11 a light source having an optical characteristic in which illumination light becomes central darkening in order to decrease the luminous intensity of light which is radiated to the vicinity of the lateral viewing angle of zero degrees of the observation system as well as to increase the luminous intensity of light which is radiated to the vicinity of the backward viewing angle of 40 degrees of the observation optical system.

For example, a light source having electrodes, such as a xenon light source, is used as the light source 11 and light emerging from the light source 11 is made to pass through the condenser lens 12 and to enter the light guide 2. As a result, a luminous intensity distribution characteristic in which illumination light becomes central darkening is generated.

Also, if a light guide having a large diameter is used as the light guide 2 in such constitution, then the angles of incidence of light rays from the light source unit 1 entering the light guide 2 vary, so that the characteristic in which the central illuminance of illumination light is low varies. In such case, it is possible to generate the characteristic in which illumination light becomes central darkening, using a means which makes equal the angles of light rays slantingly incident on the light guide 2. This matter will be explained in detail using FIGS. 4(a) to (c).

The light source unit 1 reflects light approximately coming from a cathode 11a through a reflection mirror 11c (parabolic mirror) in the light source 1 to transform the light into parallel light, concentrates the light through the condenser lens 12, and makes the light form an image on the entrance-side end plane 2a on the light-source side of the light guide 2. In this case, a light ray which is a low NA component is blocked by the cathode 11a and an anode 11b on the reflection-mirror side in the light source 1, so that the central illuminance of illumination light becomes low (illumination light becomes central darkening). The central darkening remarkably occurs, especially, in a light source having a small diameter. On the other hand, the central darkening as in the light source having a small diameter does not remarkably occur in a light source having a large diameter.

This matter is explained using FIG. 4(b). A sign, a, in FIG. 4(b) denotes a light ray which is incident on the central portion of the entrance-side end plane 2a of the light guide 2 and has a large illumination angle and a high NA. A sign, b, in FIG. 4(b) denotes a light ray which travels in the direction of a light ray incident on the central portion of the entrance-side end plane 2a of the light guide 2 and is blocked by the electrodes 11a and 11b. A sign, c, in FIG. 4(b) denotes a light ray which is incident on the peripheral portion of the entrance-side end plane 2a of the light guide 2 and has a large illumination angle and a high NA. A sign, d, in FIG. 4(b) denotes a light ray which is incident on the peripheral portion of the entrance-side end plane 2a of the light guide 2 and is blocked by the electrode (cathode) 11b. A sign, e, in FIG. 4(b) denotes a light ray which is incident on the peripheral portion of the entrance-side end plane 2a of the light guide 2 and is parallel to an optical axis.

It is clear from FIG. 4(b) that the small diameter of the light guide 2, or the small image height of the condenser lens 12 makes the influence of the eclipse by the electrodes 11a and 11b large, so that it is possible to make the luminous intensity distribution have an apparent central darkening characteristic. Besides, even in the case where the light guide 2 has a large diameter, it is possible to control the central darkening characteristic by the use of the means which makes equal the angles of light rays slantingly incident on the light guide 2, as shown in FIG. 4(c).

The bright point (light source) in the cathode 11a is ideally regarded as a point which has no dimension. However, the bright point actually has some degree of dimension. As a result, light which is reflected by the reflection mirror 11c includes not only light rays which is parallel to the optical axis, as shown by the signs a and b in FIG. 4(b), but also light rays which is not parallel to the optical axis, as shown by the signs c and d in FIG. 4(b), so that the light becomes a bundle of light rays which has some degree of dimension.

That is to say, when the diameter of the light guide 2a is large, light ray which travels in the direction of a light ray incident on the peripheral portion of the entrance-side end plane 2a of the light guide 2 and is blocked by the electrode 11b in the reflection mirror 11c (the light ray shown by the sign, d, in FIG. 4(b)) is made to slant to the optical axis at some degree of angle.

As a result, a light which is incident on the peripheral portion of the entrance-side end plane 2a of the light guide 2 in parallel to the optical axis (the light ray shown by the sign, e, in FIG. 4(b)) somewhat differs from a light ray which is blocked in the central portion of the entrance-side end plane 2a of the light guide 2, in angle of incidence, so that the luminous intensity distribution characteristic is equalized as the whole of the light guide 2 and does not have an extreme central darkening characteristic.

In such case, a field lens 13 which includes a concave lens is allocated to the entrance-side end plane 2a of the light guide 2 to equalize the angle of incidence of the light ray, d, blocked by the electrode 11b with the angle of incidence of the light ray, b, blocked by the electrodes 11a and 11b on the optical axis. As a result, it is possible to achieve a central darkening characteristic in the whole of the light guide 2.

Also, a viewing mask 14 which blocks a light ray with a small angle of incidence may be provided in a light source unit 1′ as a means except the means of equalizing the angles of light rays slantingly incident on the light guide 2, as shown in FIG. 4(d). This manner makes it possible to block a light ray in the vicinity of the optical axis to secure the central darkening characteristic, even though the light guide 2 has a large diameter.

FIG. 5 is a graph showing one example of the luminous intensity distribution characteristic having the central darkening characteristic when the light source 1 is combined with the light guide 2.

The reflection means 3 is arranged on the exit-side of the light guide 2, is approximately ring-shaped (is approximately horseshoe-shaped in FIG. 6), and is provided with a plurality of reflection planes 3a in the circumferential direction of the endoscope, as shown in FIG. 6, where each of the reflection planes 3a has an angle of reflection of 45 degrees to light which has a luminous intensity distribution characteristic having the central darkening characteristic as shown in FIG. 5, as shown in FIG. 4(e). In FIG. 4(e), the sign, 2b, denotes the exit-side end plane of the light guide 2.

Besides, the exit-side end plane 2b of the light guide 2 branches into a plurality of end planes according to the number of the reflection planes 3a of the reflection means 3.

In the illumination optical system for endoscopes according to the first embodiment having such constitution, light from the light source unit 1 enters the entrance-side end plane 2a of the light guide 2. And, light emerging from the exit-side end plane 2b of the light guide 2 is reflected by the reflection plane 3a of the reflection means 3 to be radiated to a predetermined area ranging from the lateral direction to the backward direction with respect to the longitudinal direction of the endoscope. In this case, the light from the light source unit 1 which has entered the light guide 2 has a luminous intensity distribution characteristic with the central darkening characteristic, so that it is possible to decrease the intensity of a light ray which is radiated in the direction of a zero degrees angle to the lateral direction of the endoscope (or at an angle of zero degrees to the direction perpendicular to the longitudinal direction of the endoscope) as well as to increase the intensity of a light ray which is radiated backward at an angle of 40 degrees (or at an angle of 40 degrees to the direction perpendicular to the longitudinal direction of the endoscope and in the backward direction with respect to the longitudinal direction of the endoscope).

Besides, the reflection plane 3a of the reflection means 3 shown in FIG. 4(e) is slanted in such a way the reflection angle is an angle of 45 degrees. However, if it is possible to make the intensity of light radiated to the vicinity of the lateral direction with respect to the longitudinal direction of the endoscope lower than the intensity of light radiated to a predetermined backward area with respect to the longitudinal direction of the endoscope, then the reflection angle may be lager than 45 degrees, and, for example, the reflection plane 3a may be slanted in such a way that the reflection angle is an angle of 50 degrees.

When the reflection angle of the reflection plane 3a changes from 45 degrees to 50 degrees, the reflection optical axis can be slanted by 10 degrees in the backward direction with respect to the longitudinal direction of the endoscope. As a result, the luminous intensity distribution characteristic in the case of the reflection plane 3a having an reflection angle of 50 degrees corresponds to a distribution characteristic which is obtained by shifting the whole of the luminous intensity distribution characteristic shown in FIG. 5 along the horizontal axis in FIG. 5 by +10 degrees. Accordingly, the slant of the reflection plane 3a of the reflection means 3 at an angle of 50 degrees can make the intensity of a light ray radiated at an angle of 40 degrees backward with respect to the direction perpendicular to the longitudinal direction of the endoscope equal to the intensity of a light ray radiated at an angle of 30 degrees backward with respect to the direction perpendicular to the longitudinal direction of the endoscope in the case of the slant of the reflection plane 3a at an angle of 45 degrees. As a result, an amount of light of about 50% is obtained in a luminous intensity distribution characteristic for a planer object, and it is possible to observe a bright observation image.

Also, it is preferred that the illumination angle in the luminous intensity distribution characteristic is extended more than that shown in FIG. 5, because it is possible to make the backward area brighter to observe the area.

In order to extend the relative illumination angle with the luminous intensity distribution characteristic holding the central darkening characteristic which is obtained by combining the light guide 2 with the light source unit 1, the above-described illumination optical system should be an illumination optical system in which a magnification is changed without changing the intensity distribution in pupil, or pupil aberration is removed well.

Or, for a simpler constitution, an additional light diffusing means such as a concave lens or glass beads may be arranged on the reflection side of the reflection plane 3a of the reflection means 3 to extend the luminous intensity distribution characteristic.

Or, as shown in FIG. 4(f), the reflection plane 3a of the reflection means 3 may be formed as a convex surface to have a light diffusion effect. Besides, in this case, a too strong degree of diffusion prevents the luminous intensity density characteristic from having the central darkening characteristic. Accordingly, the convex surface of the reflection plane 3a should be formed to such an extent that the convex surface has a light diffusion effect while holding the central darkening characteristic.

### The Second Embodiment

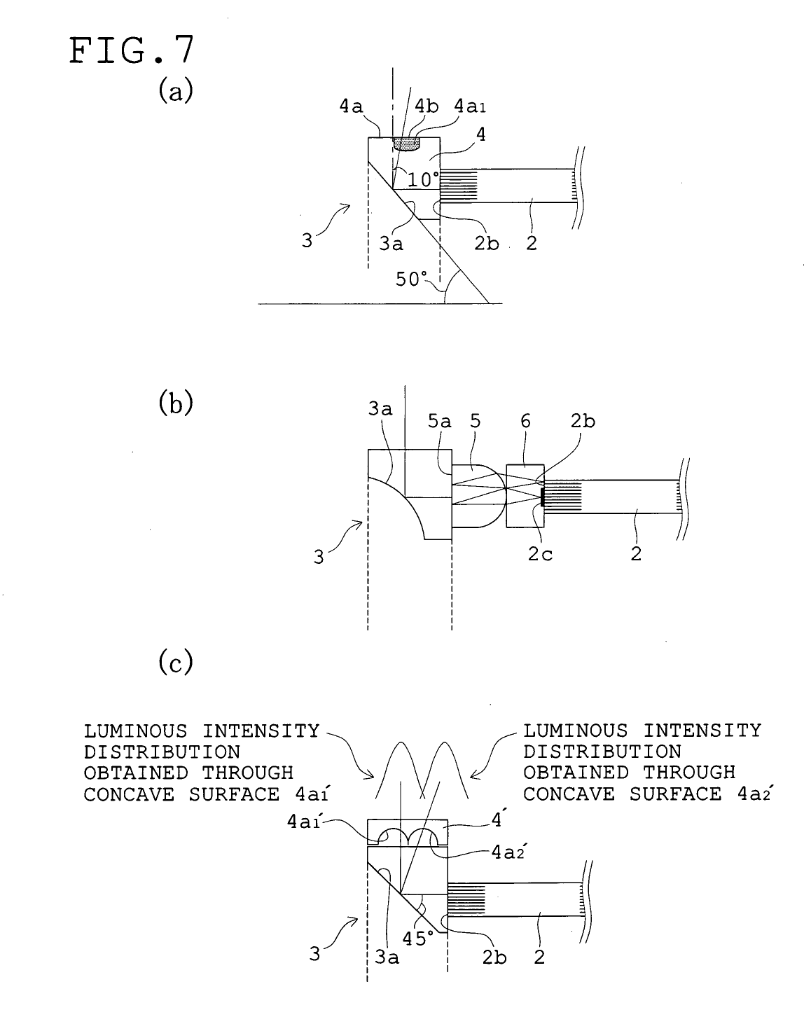

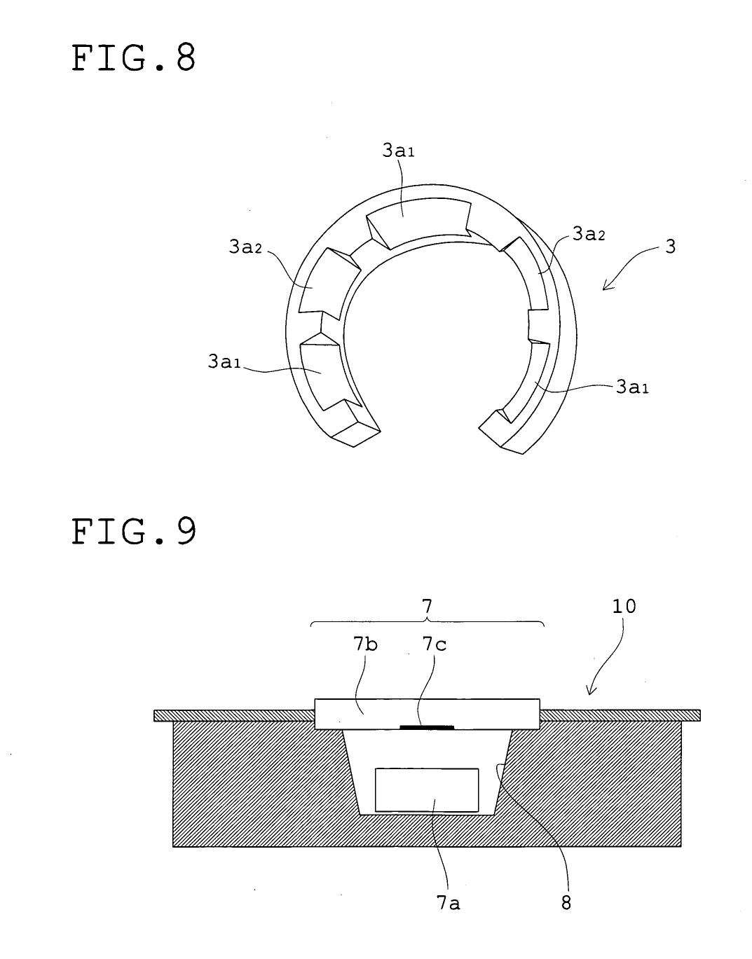

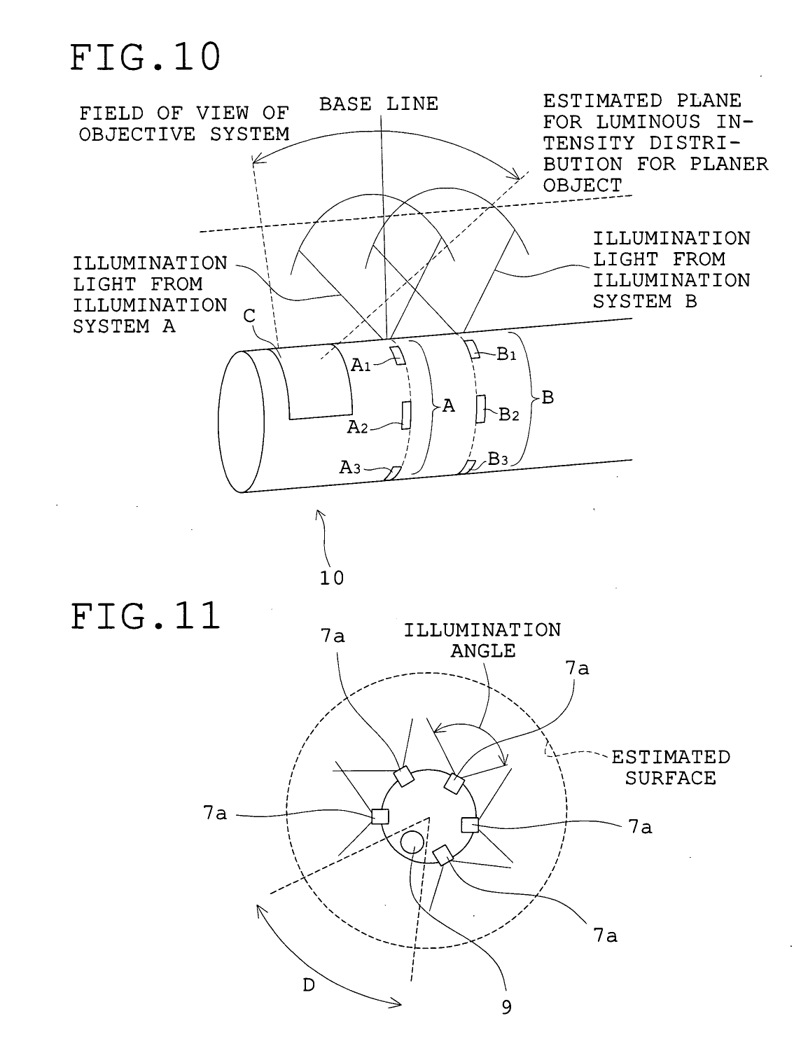

FIG. 7 is an explanatory view showing the schematic formation of an important part of an illumination optical system for endoscopes according to a second embodiment of the present invention, (a) is a view showing one example of an illumination lens which is used as one component for the illumination optical system for endoscopes according to the second embodiment, (b) is a view showing one example of variations of the illumination optical system according for endoscopes according to the second embodiment, and (c) is a view showing another example of variations of the illumination optical system for endoscopes according to the second embodiment. FIG. 8 is an explanatory view showing the schematic formation of an important part for still another example of variations of the illumination optical system for endoscopes according to the second embodiment of the present invention. FIG. 9 is an explanatory view showing the schematic formation of an important part for still another example of variations of the illumination optical system for endoscopes according to the second embodiment of the present invention. FIG. 10 is an explanatory view showing the schematic formation of an important part for still another example of variations of the illumination optical system for endoscopes according to the second embodiment of the present invention.

In the illumination optical system for endoscopes according to the second embodiment, the following formation is adopted as a means for decreasing the luminous intensity of a light ray which is radiated to the vicinity of the lateral-directional viewing angle of zero degrees of the observation system as well as increasing the luminous intensity of a light ray which is radiated to the vicinity of the backward viewing angle of 40 degrees of the observation system without using for the light source unit a light source having an optical characteristic having the central darkening characteristic.

The illumination optical system for endoscopes shown in FIG. 7(a) is provided with a lens along the circumferential direction of the endoscope, as one example of such means, where the lens is provided with a light blocking means in the central portion of the exit-side end surface of the lens.

In a more detailed explanation, the illumination optical system for endoscopes shown in FIG. 7(a) comprises a light source unit which has a luminous intensity distribution characteristic without the central darkening characteristic and is omitted in the drawings, a light guide 2 which light from the light source unit enters, and an illumination lens 4 which is provided with a reflection surface 3a reflecting light from the light guide 2 in the lateral direction of the endoscope. Besides, the reflection means 3 which is approximately ring-shaped is provided with a plurality of reflection planes 3a, as shown in FIG. 6. The number of the illumination lenses 4 corresponds to the number of the reflection planes 3a of the reflection means 3, and the illumination lens 4 is joined to or integrated with the reflection plane 3a. Also, the exit-side end plane 2b of the light guide 2 branches into a plurality of end planes according to the number of the reflection planes 3a of the reflection means 3.

The reflection plane 3a is slanted so that a light ray is reflected at an angle of reflection of 50 degrees by the reflection plane 3a.

A concave surface 4a₁ is formed in the central portion of the exit-side end plane 4a of the illumination lens 4, and the concave surface 4a₁ is provided with a light-blocking mask 4b.

And, in the illumination optical system for endoscopes shown in FIG. 7(a) and having such formation, the light-blocking mask 4b blocks a light ray which is reflected by the reflection plane 3a to travel toward the central portion of the exit-side end plane 4a and the angle of which is gentle (for example, a light ray which slants backward at an angle of 10 degrees to the direction perpendicular to the longitudinal direction of the endoscope in FIG. 7(a)), so that the central darkening characteristic is achieved, and the luminous intensity distribution characteristic is shifted backward by 10 degrees and, as a result, the intensity of a light ray radiated backward is increased.

Besides, the concave surface 4a₁ of the illumination lens 4 may be formed as a grained surface instead of the light-blocking mask 4b, so that light is diffused and an amount of light in the central portion are relatively blocked.

Also, in another example, the illumination optical system for endoscopes shown in FIG. 7(b) comprises a light source unit which has a luminous intensity distribution characteristic without the central darkening characteristic and is omitted in the drawings, a light guide 2 which light from the light source unit enters, a condenser lens 5, and a reflection means 3 which is provided with a reflection plane 3a. Besides, the approximately ring-shaped reflection means 3 is provided with a plurality of the reflection planes 3a, as shown in FIG. 5. Also, the exit-side end plane 2b of the light guide 2 branches into a plurality of end planes according to the number of the reflection planes 3a of the reflection means 3.

The light guide 2 is provided with a light-blocking mask or a core bar in the central portion of the exit-side end plane 2b as a light blocking means 2c so that light emerging from the exit-side end plane 2b has an illumination position distribution characteristic in which the distribution of illumination positions is annular.

Also, the condenser lens 5 is formed in such a way that the vicinity of the exit-side end plane 5a of the condenser lens 5 corresponds to a pupil position, and light formed through the light guide 2 to have an annular illumination position distribution characteristic is changed into light having an angle distribution characteristic.

Also, the reflection plane 3a is formed as a convex surface and has the same light diffusion effect as a concave lens. And, light having the angle distribution characteristic transformed through the condenser lens 5 is reflected to be diffused, through the reflection plane 3a of the reflection means 3. As a result, light emitting in the lateral direction is made to have a luminous intensity distribution with the central darkening characteristic. Besides, the reference numeral, 6 in FIG. 7(b) denotes a transparent glass member for adjusting the distance between the condenser lens 5 and the light guide 2 to make the position of the rear-side focal point of the condenser lens 5 correspond with the exit-side end plane 2b of the light guide 2. In the formation shown in FIG. 7(b), the luminous intensity distribution characteristic with the central darkening characteristic is obtained in the exit-side end plane 5a of the condenser lens 5.

In addition, in yet another example, as shown in FIG. 7(c), the illumination optical system may be provided with a lens (illumination lens 4′) in the circumference direction of the endoscope, where the lens has a surface having two optical powers and has a luminous intensity distribution characteristic in which the luminous intensity distribution characteristic in the longitudinal direction has a central darkening characteristic by the superposition of luminous intensity distribution characteristics in the surface having the two optical powers.

The illumination optical system for endoscopes shown in FIG. 7(c) comprises a light source unit which has a luminous intensity distribution characteristic without the central darkening characteristic and is omitted in the drawings, a light guide 2 which light from the light source unit enters, a reflection means 3 which is provided with a reflection plane 3a, and an illumination lens 4. Besides, the reflection plane 3a is slanted so that a light ray is reflected at an angle of reflection of 45 degrees by the reflection plane 3a. Also, the approximately ring-shaped reflection means 3 is provided with a plurality of the reflection planes 3a, as shown in FIG. 6. Also, the exit-side end plane 2b of the light guide 2 branches into a plurality of end planes according to the number of the reflection planes 3a of the reflection means 3.

The illumination lens 4′ is provided with two concave surfaces 4a₁′ and 4a₂′ on the entrance side thereof and along the longitudinal direction of the endoscope. And, when the luminous intensity distribution characteristics of the concave surfaces 4a₁′ and 4a₂′ are superposed, the intensity of light which passes between the two concave surfaces 4a₁′ and 4a₂′ becomes low, so that it is possible to obtain a luminous intensity distribution characteristic having a central darkening characteristic in the whole of the illumination lens 4.

Also, in still another example, the illumination optical system may has a formation in which the reflection plane of the reflection means is provided with a film the reflection characteristic of which varies depending on angle of incidence, however the formation is omitted in the drawings. In a detailed explanation, when an angle of reflection is 45 degrees in the reflection plane, the reflection plane is provided with a film having a reflection characteristic in which light incident on the reflection plane at an angle of 45 degrees has a low light intensity after the light is reflected by the reflection plane. This manner also makes it possible to achieve a luminous intensity distribution characteristic having a central darkening characteristic.

Also, in yet another example, as shown in FIG. 8, the illumination optical system may be provided with a plurality of reflection planes, 3a₁ and 3a₂ in the circumferential direction of the endoscope in such a way that reflection surfaces adjacent to each other in a plurality of the reflection surfaces, 3a₁ and 3a₂ differ form each other in angle of reflection so that the luminous intensity distribution characteristic between the reflection surfaces adjacent to each other has a central darkening characteristic, in order to change a degree of the distribution of luminous intensities.

In this case, the luminous intensity distribution characteristics in the circumferential direction of the endoscope is made in such a way that the luminous intensity distribution characteristics of the reflection surfaces the angles of reflection of which are the same as one another have no central darkening characteristic and the luminous intensity distribution characteristic between reflection surfaces which is adjacent to each other and differ from each other in angle of reflection at which light is reflected has a central darkening characteristic.

Also, in still another example, as shown in FIG. 9, the illumination optical system may be provided with a plurality of LDE light sources in the circumferential direction of the endoscope, each of the LED light sources is provided with a light-attenuating means in the area of the central portion in front of the light-emitting means and along the circumferential direction of the endoscope, and the light-attenuating means attenuates a light component having a small illumination angle.

The illumination optical system for endoscopes shown in FIG. 9 is provided with a plurality of LED light sources 7 in the circumferential direction of the endoscope, where each of the LED light sources 7 includes a LED 7a, a transparent protection member 7b, and a light-blocking member 7c.

The LED 7a is provided in each of a plurality of grooves 8 which is formed along the circumferential direction of the top portion 10 of the endoscope. Besides, only the cross section of one of the LED light sources 7 is shown in FIG. 9 for convenience. Each of the transparent protection members 7b is provided over the groove 8 to protect each LED 7a.

The light-blocking member 7c is made of an evaporated chromium film and is provided in the area of the center of the transparent protection member 7b and along the circumferential direction of the endoscope.

The LED 7a has a luminous intensity distribution characteristic without a central darkening characteristic but has a Lambertian luminous characteristic. Accordingly, in order to make light from the LED 7 light source have a luminous intensity distribution characteristic with a central darkening characteristic in the example shown in FIG. 9, a luminous intensity distribution characteristic with a central darkening characteristic is given to the light from the LED light source 7 by blocking a light ray having a small illumination angle in the area of the center in front of the LED 7a and along the circumferential direction of the endoscope.

Also, in yet another example, as shown in FIG. 10, the illumination optical system for endoscopes may be composed of two illumination systems A and B which are arranged along the longitudinal direction and illuminate a lateral surrounding area of the endoscope, where the backward-side illumination system B of the two illumination systems A and B is made to have a high intensity of light so that a luminous intensity distribution characteristic with a central darkening characteristic is made. Besides, in this case, the position corresponding to an angle of zero degrees as the base angle in a luminous intensity distribution characteristic should be a position of each of emitting optical axes in the forward-side illumination system A which is arranged nearest to the objective optical system, where the angular direction of each of the emitting optical axes is the direction perpendicular to the longitudinal direction of the endoscope and the position of each of the emitting optical axes becomes the brightest in image information. Signs, A₁, A₂, A₃, B₁, B₂, and B₃ denote emitting members which are provided along the circumferential direction of the top portion 10 of the endoscope in the illumination systems A and B, and sign, C denotes an observation window in the objective optical system, in FIG. 10.

### The Third Embodiment

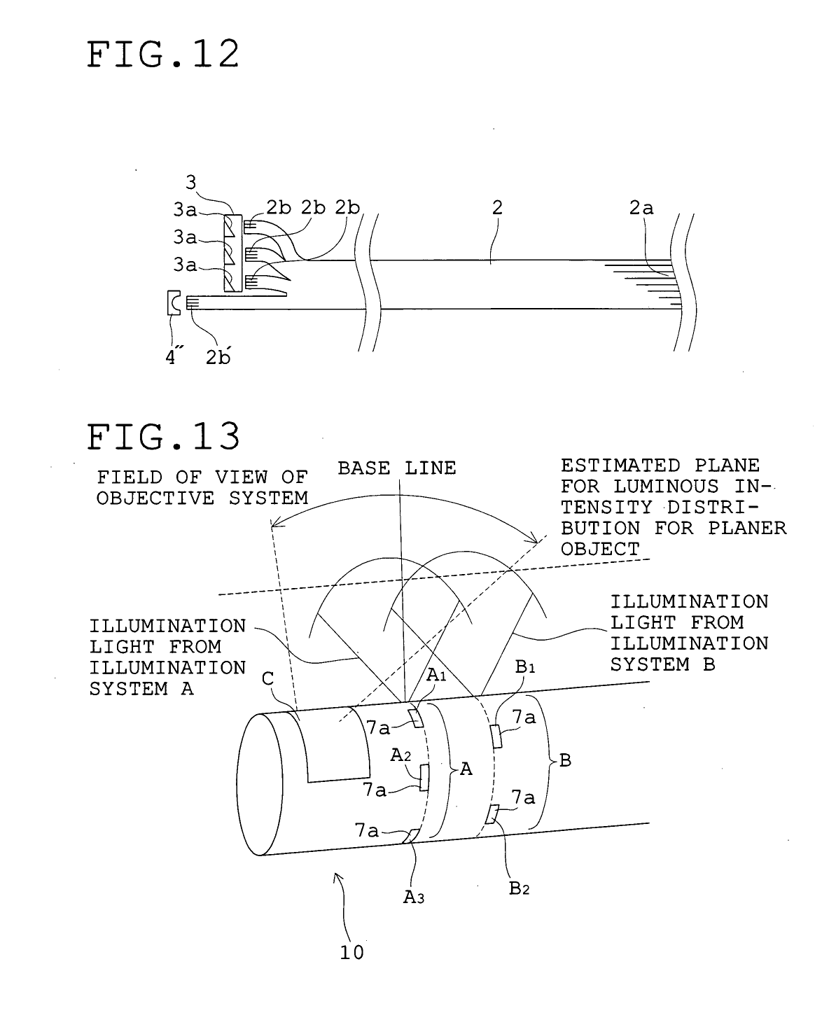

FIG. 12 is an explanatory view showing the formation of an important part of an illumination optical system for endoscopes according to a third embodiment of the present invention.

The illumination optical system according to the third embodiment comprises a light source unit which is omitted in drawings, a light guide 2 which light from the light source unit enters, and a reflection means 3 which is provided with reflection planes 3a along the circumferential direction of the endoscope, as shown in FIG. 6. The light guide 2 has one end plane 2a on the entrance side and the exit-side end which branches into a plurality of end planes. One of the end planes into which the light guide 2 branches on the exit side is used as an end plane 2b′ for forward illumination and the other end planes are used as end planes 2b for illumination toward the lateral surrounding area or backward illumination. Besides, the numeral reference, 4″ in FIG. 12 denotes an illumination lens for forward illumination.

For light having a luminous intensity distribution characteristic with a central darkening characteristic as shown in FIG. 5 which is generated, for example, by combining the light source with the light guide in the illumination optical system for endoscopes according to the first embodiment, the luminous intensity distribution of the light is extended through a diffusing means such as a concave lens. As a result, luminous intensity distribution characteristics which have a central darkening characteristic and depend on angle are mixed, so that it is possible to transform the light having the luminous intensity distribution characteristic with the central darkening characteristic into light having a luminous intensity distribution characteristic without a central darkening characteristic as shown in FIG. 3.

Accordingly, the illumination optical system for endoscopes according to the third embodiment is formed in such a way that: the light guide 2 has one end plane 2b′ on the entrance side and the exit-side end which branches into a plurality of the end planes 2b, as shown in FIG. 12; and one end plane 2b′ is used for illuminating light forward and the other end planes 2b are used for illuminating light toward the lateral surrounding area. This manner makes it possible: to exploit a luminous intensity distribution characteristics having a central darkening characteristic in each of the above-described examples, in the case where the illumination optical system is used as an illumination optical system which illuminates light to an observation area of the objective optical system which observes the lateral surrounding area of the endoscope by using the system by way of the exit-side end planes 2b of the light guide 2 and the reference means 3; and to transform light from the light guide 2 into light having a luminous intensity distribution characteristic without a central darkening characteristic to widen an illumination angle depending on a viewing angle of the objective optical system which observes a forward area, by using as the illumination lens 4″ a lens such as a concave lens which has a diffusion effect, in the case where the illumination optical system is used as the second illumination optical system which illuminates light to an observation area of the objective optical system which observes the forward area of the endoscope by using the system by way of the exit-side end plane 2b′ of the light guide 2 and the illumination lens 4″.

### The Fourth Embodiment

An illumination optical system for endoscopes according to the fourth embodiment is an illumination optical system for endoscopes: which is combined with an objective optical system in using the illumination optical system, where the objective optical system makes it possible to observe a predetermined area at least ranging from the lateral direction to the backward direction and across the range of 180 degrees or more in the circumferential direction of the endoscope; and which comprises an illumination optical system capable of illuminating light to the area which the objective optical system can observe. In addition, the illumination optical system according to the fourth embodiment is formed in such a way that a luminous intensity distribution characteristic in the longitudinal direction of the endoscope has luminous intensities of 85% or more as a luminous intensity distribution characteristic for a spherical object which is normalized by the maximum intensity in the range of the illuminating angles corresponding to the observation viewing angles of the objective optical system.

Illumination optical systems for endoscopes of the present invention including the illumination optical systems for endoscopes of the above-described embodiments differ from a conventional illumination optical system for endoscopes, and is illumination optical systems which are combined with an observation system capable of observing the area ranging from the lateral direction to the backward direction in using the illumination optical system. In a more detailed explanation, the illumination optical systems for endoscopes of the present invention are an illumination optical system for endoscopes: which is combined with an objective optical system in using the illumination optical system, where the objective optical system can observe a predetermined area at least ranging at least from the lateral direction to the backward direction and across the range of 180 degrees or more in the circumferential direction of the endoscope; and which comprises an illumination optical system capable of illuminating light to the area which the objective optical system can observe.

That is to say, the illumination optical systems for endoscopes of the present invention are formed in such a way that the illumination optical systems can be recomposed into illumination systems which differ from one another in state of luminous intensity distribution in accordance with the observed area and observed direction of an observation system which is used for observation.

For example, in the case where an observation system has a backward viewing area of only 20 degrees, the light intensity of 70% in a luminous intensity distribution for a planer object can be obtained even though a illumination optical system having a luminous intensity distribution characteristic without a central darkening characteristic as shown in FIG. 3 is used, and the influence of uneven illumination is considered to be small even as compared with the light intensity of 100% at the illumination angle of zero degrees. Accordingly, in such case where the observation system has a narrow area of backward viewing fields, it is considered possible to give sufficient brightness to an object in a backward observation area, even though the luminous intensity distribution characteristic of the illumination optical system has no central darkening characteristic.

However, the case where such condition is satisfied is limited to the case where: the viewing area of an observation system is persistently limited to a predetermined area which ranges from the lateral direction to the backward direction and is not so wide; and the observation system is also combined with an illumination optical system having a luminous intensity distribution characteristic which makes it possible to obtain considerable degree of light intensity in the predetermined area.

### The Fifth Embodiment

FIG. 11 is a conceptual view showing one example of the positional relation between light sources and the illumination angles when an endoscope which is provided with the illumination optical system for endoscopes according to the present invention is viewed from the front.

Endoscopes to which illumination optical systems for endoscopes of the present invention explained in each of the above-described embodiments are applicable are provided with a treatment channel aperture 9 through which a treatment means for collecting a part of a living body passes, as shown in FIG. 11. As a result, it is impossible to observe the whole of the circumference of 360 degrees with respect to the circumferential direction of the endoscope, and a blind spot D occurs in a part of the observation system.

The endoscope shown in FIG. 11 is formed in such a way that five light sources 7a such as LED are arranged at intervals of 60 degrees so that it is possible to observe an area of approximately 300 degrees in the inside surface of a lumen such as a large intestine.

In this case, a surface in which a luminous intensity distribution is estimated is the inside surface of a lumen, surrounds the endoscope, and is shaped like a cylinder. However, it is desired that the luminous intensity distribution characteristic in the circumferential direction of the endoscope has no unevenness and is uniform in the cylinder-shaped estimated surface. On the other hand, it is desired that the luminous intensity distribution characteristic in the longitudinal direction of the endoscope has a central darkening characteristic in order to decrease a light intensity at an angle of zero degrees to the lateral direction and to increase a light intensity on the backward side of the endoscope in the cylinder-shaped estimated surface, as described above.

Accordingly, an illumination optical system for endoscopes according to the fifth embodiment is formed in such a way that: the illumination optical system can illuminate o a predetermined area at least ranging from the lateral direction to the backward direction across 180 degrees or more in the circumferential direction of the endoscope, where the area can be observed by the objective optical system; and the luminous intensity distribution characteristic in the longitudinal direction of the endoscope differs from the luminous intensity distribution characteristic in the circumferential direction of the endoscope in the illumination optical system.

Next, its specific example will be shown.

As one example of the specific example, the illumination optical system is provided with a plurality of light sources 7a in the circumferential direction of the endoscope in such a way that: the light sources 7a have a luminous intensity distribution characteristic with a central darkening characteristic; and the luminous intensity distribution characteristic in the circumferential direction of the endoscope maintains predetermined light intensities by the superposition of the luminous intensities of light sources adjacent to each other in the light sources 7a. This constitution will be explained in more detail using specific numerical values.

In a light emitting source having a luminous intensity distribution characteristic for spherical object as shown in FIG. 5, a light intensity becomes 30% at an illumination angle of about 35 degrees. Accordingly, when the light sources 7a which have such luminous intensity distribution characteristic are arranged along the circumferential direction of the endoscope at intervals of an angle of 70 degrees, light intensity between one light source 7a and another light source 7a becomes about 60% by the addition of light intensities of 30% at the illumination angle of an angle of 35 degrees in each of the light sources 7a. As a result, the light intensities range from 60% to 100% across the whole of illumination area in the circumferential direction of the endoscope and it is possible to obtain a luminous intensity distribution characteristic generating an approximately uniform amount of light which does not affect observation.

The use of the superposition of the luminous intensities of light sources adjacent to each other in such manner can make the luminous intensity distribution characteristic in the circumferential direction of the endoscope different from the luminous intensity distribution characteristic in the longitudinal direction of the endoscope.

Also, as another example, the illumination optical system may be formed in such a way that the optical power in the longitudinal direction of the endoscopes differs from the optical power in the circumferential direction of the endoscopes in the illumination optical system. This manner makes it possible to make the luminous intensity distribution characteristics in the circumferential direction of the endoscope different from the luminous intensity distribution characteristics in the longitudinal directions of the endoscope, like the first example.

As still another example, the illumination optical system is provided with a light guide having an end plane, where the size of the end plane in the direction corresponding to the longitudinal direction of the endoscope differs from the size of the end plane in the direction corresponding to the circumferential direction of the endoscope. This manner can makes the luminous intensity distribution characteristic in the longitudinal direction of the endoscope different from the luminous intensity distribution characteristic in the circumferential direction of the endoscope, like the first example, while the optical power in the longitudinal direction of the endoscope in the illumination optical system being the same as the optical power in the circumferential direction of the endoscope in the illumination optical system.

Also, as yet another example, the illumination optical system may be provided with a diffuser element which controls a luminous intensity distribution characteristic. This manner can make the luminous intensity distribution characteristic in the longitudinal direction of the endoscope different from the luminous intensity distribution characteristic in the circumferential direction of the endoscope, like the first example.

Also, as still another example, the illumination optical system may be provided with a plurality of sets of light sources in the longitudinal direction of the endoscope, as shown in FIG. 10, where the light sources have a Lambertian characteristic and are arranged along the circumferential direction of the endoscope.

In this case, as shown in FIG. 13, it is preferred that the illumination optical system is provided with a plurality of the sets of the light sources in such a way that the light sources 7a in the sets of the light sources are staggered with the light sources of one of the sets alternating with the light sources of the other of the sets, because it is possible to maintain a desired luminous intensity distribution characteristic as well as to restrict the number of the light sources which are arranged along the circumferential direction of the endoscope to as a small number as possible. Signs, A₁, A₂, A₃, B₁, B₂, and B₃ denote the emitting members of the light sources which are provided along the circumferential direction of the top portion 10 of the endoscope in illumination systems A and B, and a sign, C denotes an observation window of the objective optical system, in FIG. 13.

According to the illumination optical systems for endoscopes which are explained in the above-described embodiments respectively, it is possible to decrease a luminous intensity of a light ray which is radiated to the vicinity of a viewing angle of the observation system of zero degrees to the lateral direction perpendicular to the longitudinal direction of the endoscope as well as to increase a luminous intensity of a light ray which is radiated to the vicinity of a backward viewing angle of 40 degrees of the observation system. As a result, when a lumen is observed by an endoscope with an illumination optical system for endoscopes which is explained in each of the above-described embodiments, it is possible to perform bright backward observation. In addition, even though the distance to an object in the lateral direction perpendicular to the longitudinal direction of the endoscope varies, it is possible to limit a brightness variation accompanying the distance variation to a small extent. Also, the illumination optical systems for endoscopes which are explained in the above-described embodiments respectively have no configuration constitution in which the illumination optical system is thick in the direction of the diameter of the endoscope, so that it is possible to secure thinness enough to observe a lumen as endoscope. As a result, it is possible to detect a backward lesion in a lumen such as large intestine with good accuracy.

Besides, an illumination optical system for endoscopes of the present invention is not limited to the constitutions which are shown in the above-described embodiments respectively. Needless to say, an illumination optical system for endoscope of the present invention may use any combination of the characteristic constitutions in the embodiments.

An illumination optical system for endoscopes of the present invention is useful, for example, for the fields of medical treatment and medical science which require an accurate detection of a backward lesion in a lumen such as large intestine.

What is claimed is:

1. An illumination optical system for endoscopes which is used by combining the illumination optical system with an objective optical system capable of observing a predetermined area at least ranging from the lateral direction to the backward direction with respect to the longitudinal direction of an endoscope and across the range of 180 degrees or more in the circumferential direction of the endoscope, and which is provided with an illumination optical system capable of radiating light to the predetermined area which can be observed by the objective optical system,
wherein illumination light becomes central darkening along the longitudinal direction of the endoscope and across the range of 180 degrees in the circumferential direction of the endoscope which can be observed by the objective optical system, and
the illumination optical system has a luminous intensity distribution characteristic in which the central-darkening illumination light is radiated to the vicinity of the lateral direction with respect to the longitudinal direction of the endoscope.

2. An illumination optical system for endoscopes according to claim 1, wherein the luminous intensity distribution characteristic along the longitudinal direction of the endoscope satisfies the following condition in the illumination optical system:
I₀≦Iθ
where, I denotes the intensity of light which is radiated by the illumination optical system with a luminous intensity distribution for a spherical object at a predetermined angle to the direction perpendicular to the longitudinal direction of the endoscope within the range of the field of view which can be observed by the objective optical system, on the basis of a light radiation plane of the illumination optical system, I₀ denotes the intensity of light which is radiated at an angle of zero degrees to the direction perpendicular to the longitudinal direction of the endoscope by the illumination optical system with a luminous intensity distribution for a spherical object, and Iθ denotes the intensity of light which is radiated backward at an angle of θ degrees to the direction perpendicular to the longitudinal direction of the endoscope by the illumination optical system with a luminous intensity distribution for a spherical object.

3. An illumination optical system for endoscopes according to claim 2, further comprising a second illumination optical system which is used by combing the second illumination optical system with an objective optical system by which forward observation can be performed and which can radiate light forward,
wherein the second illumination optical system has a luminous intensity distribution characteristic in which illumination light does not become central darkening along the longitudinal direction of the endoscope.

4. An illumination optical system according to claim 1, wherein a luminous intensity distribution characteristic in the longitudinal direction of the endoscope is different from a luminous intensity distribution characteristic in the circumferential direction of the endoscope.

5. An illumination optical system for endoscopes according to claim 1, wherein the luminous intensity distribution characteristic along the longitudinal direction of the endoscope satisfies the following condition in the illumination optical system:
I₀≦I₄₀
where, I denotes the intensity of light which is radiated by the illumination optical system with a luminous intensity distribution for a spherical object at a predetermined angle to the direction perpendicular to the longitudinal direction of the endoscope within the range of the field of view which can be observed by the objective optical system, on the basis of a light radiation plane of the illumination optical system, I₀ denotes the intensity of light which is radiated at an angle of zero degrees to the direction perpendicular to the longitudinal direction of the endoscope by the illumination optical system with a luminous intensity distribution for a spherical object, and I₄₀ denotes the intensity of light which is radiated backward at an angle of 40 degrees to the direction perpendicular to the longitudinal direction of the endoscope by the illumination optical system with a luminous intensity distribution for a spherical object.

6. An illumination optical system for endoscopes according to claim 1, wherein the luminous intensity distribution characteristic along the longitudinal direction of the endoscope satisfies the following condition in the illumination optical system:
I₀≦I₅₀
where, I denotes the intensity of light which is radiated by the illumination optical system with a luminous intensity distribution for a spherical object at a predetermined angle to the direction perpendicular to the longitudinal direction of the endoscope within the range of the field of view which can be observed by the objective optical system, on the basis of a light radiation plane of the illumination optical system, I₀ denotes the intensity of light which is radiated at an angle of zero degrees to the direction perpendicular to the longitudinal direction of the endoscope by the illumination optical system with a luminous intensity distribution for a spherical object, and I₅₀ denotes the intensity of light which is radiated backward at an angle of θ degrees to the direction perpendicular to the longitudinal direction of the endoscope by the illumination optical system with a luminous intensity distribution for a spherical object.

7. An illumination optical system for endoscopes according to claim 2 comprising a light source unit having a luminous intensity distribution characteristic in which illumination light becomes central darkening,
a light guide which light from the light source unit enters, and
a reflection means which is approximately shaped like a ring and is provided with a reflection surface along the circumferential direction of the endoscope,
wherein the reflection surface reflects light emerging from the light guide, at the angle of reflection of 45 degrees or more.

8. An illumination optical system for endoscopes according to claim 2 comprising
a light source unit having a luminous intensity distribution characteristic in which illumination light becomes central darkening,
a light guide which light from the light source unit enters, and
a reflection means which is approximately shaped like a ring and is provided with a reflection surface along the circumferential direction of the endoscope,
wherein the reflection surface reflects light emerging from the light guide, at the angle of reflection of approximately 50 degrees.

9. An illumination optical system for endoscopes according to claim 7 comprising a means for making the angle-of-incidence characteristics of light rays from the light source unit incident on the light guide the same characteristic.

10. An illumination optical system for endoscopes according to claim 9, wherein the means for making the angle-of-incidence characteristics of the light rays the same characteristic is a concave lens which is arranged on the entrance side of the light guide.

11. An illumination optical system for endoscopes according to claim 2, wherein the light source unit is provided with a field-of-view mask blocking a light ray the angle of incidence of which is small.

12. An illumination optical system for endoscopes according to claim 7 comprising a variable magnification optical system having a function of removing pupil aberration.

13. An illumination optical system for endoscopes according to claim 7 comprising a light diffusing means on the reflection side of the reflection surface.

14. An illumination optical system for endoscopes according to claim 7, wherein the reflection surface has the diffusion effect while the luminous intensity distribution characteristic in which illumination light becomes central darkening along the longitudinal direction of the endoscope is held.

15. An illumination optical system for endoscopes according to claim 14, wherein the reflection surface is formed as a convex surface.

16. An illumination optical system for endoscopes according to claim 2 comprising a lens along the circumferential direction of the endoscope, wherein the lens is provided with a light blocking means in the central portion of the end surface on the exit side thereof.

17. An illumination optical system for endoscopes according to claim 2 comprising a light guide and a condenser lens along the circumferential direction of the endoscope, wherein the light guide is provided with a light blocking means in the central portion of the end surface on the exit side thereof and the condenser lens is arranged on the exit side of the light guide.

18. An illumination optical system for endoscopes according to claim 2 comprising a lens along the circumferential direction of the endoscope, wherein the lens has a plane having two optical powers and the luminous intensity distribution characteristics of the plane having the two optical powers are superposed so that the lens has a luminous intensity distribution characteristic in which illumination light becomes central darkening along the longitudinal direction of the endoscope.

19. An illumination optical system for endoscopes according to claim 2 comprising a reflection surface along the circumferential direction of the endoscope, wherein the reflection surface is provided with a film and the reflection characteristic of the film varies depending on angle of incidence.

20. An illumination optical system for endoscopes according to claim 2 comprising a plurality of reflection surfaces along the circumferential direction of the endoscope, wherein reflection surfaces adjacent to each other in a plurality of the reflection surfaces differ from each other in angle of reflection so that the luminous intensity distribution characteristic along the longitudinal direction of the endoscope by the reflection surfaces adjacent to each other becomes a luminous intensity distribution characteristic in which illumination light becomes central darkening.

21. An illumination optical system for endoscopes according to claim 2 comprising a plurality of LED light sources along the circumferential direction of the endoscope, wherein each of the LED light sources is provided with a light attenuating means in the middle area in front of the light emitting means and along the circumferential direction of the endoscope, and the light attenuating means reduces a light ray having a small angle in the luminous intensity distribution.

22. An illumination optical system for endoscopes according to claim 2 comprising two illumination systems which are arranged along the longitudinal direction of the endoscope and illuminate the lateral surrounding area of the endoscope, wherein the light intensities of the back-side illumination system of the two illumination systems illuminating the lateral surrounding area of the endoscope are intensified in order to make a luminous intensity distribution in which illumination light becomes central darkening.

23. An illumination optical system for endoscopes according to claim 3 comprising a light source unit and a light guide which light from the light source unit enters, wherein the light guide has one end plane on the entrance side and the exit-side end plane of the light guide branches into a plurality of end planes, and one of a plurality of the end planes is used for forward radiation of illumination light and the other end planes of a plurality of the end planes are used for radiation of illumination light in the direction perpendicular to the longitudinal direction of the endoscope or backward radiation of illumination light.

24. An illumination optical system for endoscopes according to claim 4 comprising a plurality of light sources along the circumferential direction of the endoscope, wherein the light sources have a luminous intensity distribution characteristic in which illumination light becomes central darkening, and the luminous intensity distribution characteristic in the circumferential direction of the endoscope retains a predetermined luminous intensity by the superposition of the luminous intensities of light sources adjacent to each other in a plurality of the light sources.

25. An illumination optical system for endoscopes according to claim 4, wherein the optical power in the longitudinal direction of the endoscope differs from the optical power in the circumferential direction of the endoscope in the illumination optical system.

26. An illumination optical system for endoscopes according to claim 4 comprising a light guide having an end plane the diameter in the direction corresponding to the longitudinal direction of the endoscope of which differs from the diameter of the end plane in the direction corresponding to the circumferential direction of the endoscope.

27. An illumination optical system for endoscopes according to claim 4 comprising a diffuser element which controls a luminous intensity distribution characteristic.

28. An illumination optical system for endoscopes according to claim 4 comprising a plurality of sets of light sources along the longitudinal direction of the endoscope, wherein each of the sets of the light sources consists of light sources which have a Lambertian characteristic and are arranged along the circumferential direction of the endoscope, and the light sources in sets of the light sources which are adjacent to each other are staggered with the light sources of one of the sets adjacent to each other alternating with the light sources of the other of the sets adjacent to each other.
